(19) United States
(12) Patent Application Publication
(10) Pub. No.: US 2023/0167203 A1
(43) Pub. Date: Jun. 1, 2023

(54) METHOD FOR DRYING SUGAMMADEX

(71) Applicant: MEDICHEM, S.A., Barcelona (ES)

(72) Inventors: Jordi PUIG SERRANO, Canet d'Adri (ES); Ernesto DURAN LÓPEZ, Barcelona (ES); Jordi MARKALAIN BARTA, Swieqi (MT); Joana Raquel FERREIRA DA COSTA, Marsaskala (MT)

(73) Assignee: MEDICHEM, S.A., Barcelona (ES)

(21) Appl. No.: 17/904,195

(22) PCT Filed: Jan. 13, 2021
(86) PCT No.: PCT/EP2021/050520

§ 371 Date: Aug. 12, 2022

(30) Foreign Application Priority Data
Feb. 28, 2020 (EP) 20382142.6

(51) Int. Cl. C08B 37/16; A61K 31/724

(52) U.S. Cl. CPC C08B 37/0012; A61K 31/724

(57) ABSTRACT

This invention relates to a process for preparing Sugammadex or its salts, preferably Sugammadex sodium, with a low content of organic solvents, preferably water-miscible organic solvents, more preferably ethanol, 2-propanol and/or acetone. The process comprises exposing Sugammadex or its salts, preferably Sugammadex sodium, to a medium with a high relative humidity.

This invention relates to a process for preparing 6-per-deoxy-6-per-(2-carboxyethyl)thio-y-cyclodextrin or a salt thereof with low contents of organic solvents.

### BACKGROUND OF THE INVENTION

Sugammadex is the international commonly accepted non-proprietary name (INN) for 6-per-deoxy-6-per-(2-carboxyethyl)thio-y-cyclodextrin, and has an empirical formula of C₇₂H₁₁₂O₄₈S₈ and a molecular weight of 2002.18 g/mol.

The octa-sodium salt of Sugammadex (compound I), hereinafter referred to as Sugammadex sodium, is known to be therapeutically useful in the reversal of neuromuscular blockade induced by rocuronium or vecuronium. In Europe and the United States, Sugammadex sodium is marketed under the name Bridion™.

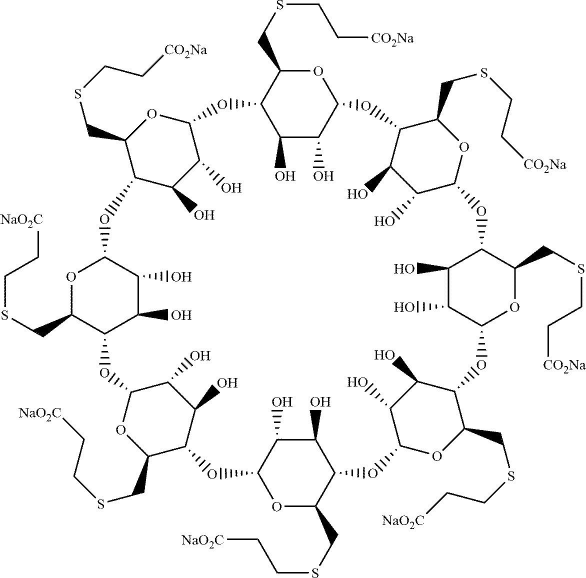

Sugammadex was described in the U.S. Pat. No. RE44,733. Specifically, Example 4 of this patent discloses the preparation of Sugammadex sodium which is isolated by filtration from a mixture of water and ethanol and afterwards dried without specifying the drying conditions. The amount of residual organic solvent, ethanol in this case, is not provided.

Several processes for preparing Sugammadex sodium are disclosed in the literature, for example WO2020058987A1, WO2020201930A1, WO2020028448A1, WO2019193198A1, WO2019002610A1, WO2019159191A1, WO2019102009A1, WO2018185784A1, WO2017163165A1, WO2017084401A1, WO2017144734A2, US2018251575A1, WO2016194001A1, WO2014125501A1 and WO2012025937A1. In these references Sugammadex sodium is dried using standard drying conditions such as drying at different temperatures or drying at different temperatures under vacuum. The amounts of residual organic solvents of the dried sugammadex sodium are not given in these references.

WO2019184773A1 discloses a method for removing gas-phase impurities, i.e. residual organic solvents, in Sugammadex sodium. The method disclosed comprises several steps: (1) dissolving the crude Sugammadex sodium in water; (2) distillation under normal or reduced pressure; and (3): freeze-drying or spray-drying the aqueous solution obtained in step (2). Therefore, the method disclosed in order to considerably reduce the presence of organic solvents in Sugammadex sodium requires several steps and some complex and expensive, non-standard evaporation techniques such as the freeze-drying or the spray-drying.

In other references of the prior art, for example US2019062459A1 and US2019062460A1, sugammadex sodium is dried using spray-drying.

CN1 10615860 discloses a process of purification which includes the steps of dissolving Sugammadex sodium containing residual solvent in water, concentrating and then drying under standard conditions (e.g. under reduced pressure). The step of concentrating involves working at temperatures of 40° C. to 70° C. and a relative vacuum degree of -0.080 to -0.098 MPa. This process is difficult to be industrially applicable because it is difficult to determine what amount of water must be removed in the concentration step in order to ensure a technically feasible isolation of Sugammadex sodium, for example by filtration, while not losing a lot of yield in view of the high solubility of Sugammadex sodium in water.

None of the references in the prior art specifies the humidity conditions during the drying process.

Toxicity or carcinogenicity of organic solvents remaining in pharmaceuticals has attracted increasing attention, and pharmaceutical administrations demand that restrictions on the amount of organic solvent residues are imposed. Therefore, in order to ensure the quality and safety of a drug product, it is important to strengthen the control of organic solvent residues in the drug substances.

Regarding the limit value of the content of the organic solvents in a drug substance, guidance is shown in ICH guideline Q3C (R6). The objective of this guideline is to recommend acceptable amounts for residual solvents in pharmaceuticals for the safety of the patient. Of course, it is desirable to have less within this range.

The inventors of the present invention have found that Sugammadex or its salts, in particular Sugammadex sodium, show a high affinity for the organic solvents, in particular for the water-miscible organic solvents, so that it has been observed that it is not possible to satisfy the limit values of the above guidelines for Sugammadex and its salts, in particular Sugammadex sodium, by using standard drying processes used in industrial manufacturing plants such as vaccum drying.

On the other hand, the processes disclosed in the prior art which provide Sugammadex or its salts, in particular Sugammadex sodium, with low contents of organic solvents, i.e. with contents in organic solvents according to the ICH guideline Q3C (R6), are complex processes which involve several steps and/or more laborious techniques, in terms of cost and time, such as lyophilization, spray-drying, freeze-drying, etc.

Therefore, there is the need to provide a more simple and at the same time industrially applicable process of removing organic solvents, preferably water-miscible organic solvents, more preferably solvents selected from the group consisting of acetic acid, acetone, acetonitrile, methanol, ethanol, 1-propanol, 2-propanol, 1 ,4-dioxane, N,N-dimethylformamide, dimethylsulfoxide and tetrahydrofuran from Sugammadex or its salts, in particular from Sugammadex sodium, in order to obtain Sugammadex or its salts, in particular Sugammadex sodium, with contents of organic solvents, in particular water-miscible organic solvents, according to ICH guideline Q3C (R6).

### BRIEF SUMMARY OF THE INVENTION

It is an object of the present invention to provide a process for removing water-miscible organic solvents selected from the group consisting of acetic acid, acetone, acetonitrile, methanol, ethanol, 1-propanol, 2-propanol, 1,4-dioxane, N,N-dimethylformamide, dimethylsulfoxide and tetrahydrofuran, from Sugammadex or its salts, preferably from Sugammadex sodium, on a large, commercial scale which allows to obtain Sugammadex or its salts, preferably Sugammadex sodium, with low contents of said water-miscible organic solvents.

The process of the present invention is a simple and industrially scalable process, characterized in that it is carried out under mild conditions.

### DETAILED DESCRIPTION OF THE INVENTION

The present invention provides a process for removing water-miscible organic solvents from Sugammadex or its salts, preferably from Sugammadex sodium, which comprises exposing Sugammadex or its salts, preferably Sugammadex sodium, to a relative humidity of 70% or higher.

The inventors of the present invention have surprisingly found that by exposing Sugammadex or its salts, preferably Sugammadex sodium, to high relative humidities, even under mild temperature conditions like room temperature, the amount of organic solvents, in particular of water-miscible organic solvents, in Sugammadex or its salts, preferably Sugammadex sodium, constantly decreased to acceptable limits according to ICH guideline Q3C (R6).

The term “removing water-miscible organic solvents from Sugammadex or its salts, preferably from Sugammadex sodium” is understood to mean the process by which the content of at least one water-miscible organic solvent in Sugammadex or its salts, preferably in Sugammadex sodium, is substantially reduced, preferably reduced to values according to ICH guideline Q3C (R6).

Examples of water-miscible organic solvents according to the present invention are acetic acid, acetone, acetonitrile, methanol, ethanol, 1-propanol, 2-propanol, 1,4-dioxane, N,N-dimethylformamide, dimethylsulfoxide and tetrahydrofuran. The ICH guideline Q3C (R6) provides for said solvents the following limits:

| Solvent | limit (ppm) |
|---|---|
| Acetic acid | 5000 |
| Acetone | 5000 |
| Acetonitrile | 410 |
| N,N-Dimethylformamide | 880 |
| Dimethylsulfoxide | 5000 |
| 1,4-Dioxane | 380 |
| Ethanol | 5000 |
| Methanol | 3000 |
| 1-Propanol | 5000 |
| 2-Propanol | 5000 |
| Tetrahydrofuran | 720 |

Sugammadex or its salts, preferably Sugammadex sodium, to which the process of the present invention is applied, comprises solid Sugammadex or its salts, preferably solid Sugammadex sodium, which contains organic solvents, in particular water-miscible organic solvents, more particularly ethanol, 2-propanol or acetone, isolated by standard isolation methods used in industrial manufacturing plants such as filtration.

In a particular embodiment Sugammadex or its salts, preferably Sugammadex sodium, to which the process of the present invention is applied, have a content of at least one water-miscible organic solvent, for example ethanol, 2-propanol or acetone, below 100,000 ppm, preferably below 50,000 ppm, more preferably below 30,000 ppm, more preferably below 25,000 ppm, even more preferably below 20,000 ppm. More preferably Sugammadex or its salts, preferably Sugammadex sodium, to which the process of the present invention is applied have a content of ethanol below 100,000 ppm, preferably below 50,000 ppm, more preferably below 30,000 ppm, more preferably below 25,000 ppm, even more preferably below 20,000 ppm.

When it is desired to use the process of the invention with Sugammadex or its salts, preferably Sugammadex sodium, having a very high content of at least one water-miscible organic solvent, preferably ethanol, 2-propanol or acetone, for example of more than 100,000 ppm, this initial content of the at least one water-miscible organic solvent, preferably ethanol, 2-propanol or acetone, may be reduced by conventional means known to the expert in field such as drying under vacuum.

The process of the present invention is preferably a drying process.

Sugammadex or its salts, preferably Sugammadex sodium, obtained by the process of the invention have a content of at least one water-miscible organic solvent, which is lower than the water-miscible organic solvent of the product to which the process of the invention is applied.

In another particular embodiment Sugammadex or its salts, preferably Sugammadex sodium, obtained by the process of the invention have an ethanol content which is lower than the ethanol content of the product to which the process of the invention is applied.

In another particular embodiment Sugammadex or its salts, preferably Sugammadex sodium, obtained by the process of the invention have a 2-propanol content which is lower than the 2-propanol content of the product to which the process of the invention is applied.

In another particular embodiment Sugammadex or its salts, preferably Sugammadex sodium, obtained by the process of the invention have an acetone content which is lower than the acetone content of the product to which the process of the invention is applied.

The Sugammadex or its salts, preferably Sugammadex sodium, which is submitted to the process for removing organic solvents of the present invention can be obtained and isolated according to any of the processes disclosed in the prior art, preferably according to the process disclosed in WO2019102009A1. For example, Sugammadex or its salts, preferably Sugammadex sodium, containg ethanol as residual organic solvent can be obtained by the processes disclosed in WO2019102009A1.

Sugammadex or its salts, preferably Sugammadex sodium, containing residual organic solvents, preferably water-miscible organic solvents, can be obtained by recrystallization or slurry of Sugammadex or its salts, preferably Sugammadex sodium, in the organic solvents, preferably the water-miscible organic solvents, or alternatively in a mixture of water and the organic solvents, preferably the water-miscible organic solvents.

The term “exposing Sugammadex or its salts, preferably Sugammadex sodium, to a certain relative humidity or higher” as used in the present invention comprises contacting Sugammadex or its salts, preferably Sugammadex sodium, totally or partially with a gaseous medium having this certain relative humidity or higher.

For example, the term “exposing Sugammadex or its salts, preferably Sugammadex sodium, to a relative humidity of 60% or higher” as used in the present invention comprises contacting Sugammadex or its salts, preferably Sugammadex sodium, totally or partially with a gaseous medium having a relative humidity of 60% or higher.

For example, the term “exposing Sugammadex or its salts, preferably Sugammadex sodium, to a relative humidity of 70% or higher” as used in the present invention comprises contacting Sugammadex or its salts, preferably Sugammadex sodium, totally or partially with a gaseous medium having a relative humidity of 70% or higher.

In a preferred embodiment of the present invention, Sugammadex or its salts, preferably Sugammadex sodium, is exposed to a relative humidity of 80% or higher.

In a preferred embodiment of the present invention, Sugammadex or its salts, preferably Sugammadex sodium, is exposed to a relative humidity of 90% or higher.

In a preferred embodiment of the present invention, Sugammadex or its salts, preferably Sugammadex sodium, is exposed to a relative humidity of from 95% to 100%.

The term “relative humidity” as used in the present invention is understood to mean the ratio of the partial pressure of water vapor to the equilibrium vapor pressure of water at a given temperature. Relative humidity is normally expressed as a percentage, which cannot exceed 100%. Relative humidities are measured with hygrometers. These humidity measurement instruments typically rely on measurements of some other quantity such as temperature, pressure, mass, a mechanical or electrical charge in a substance as moisture is absorbed and, by calibration and calculation, these measured quantities lead to a measurement of relative humidity.

The process of the present invention can be performed at a temperature from 0° C. to 100° C., preferably from 20° C. to 60° C., more preferably from 20° C. to 30° C., even more preferably about 25° C.

In a preferred embodiment of the present invention the organic solvent to be removed by the process of the invention comprises at least a water-miscible organic solvent. In a more particular embodiment, the organic solvents to be removed by the process of the invention consist of one or more water-miscible organic solvents.

The term “water-miscible organic solvent” as used herein means organic solvents which are liquid at room temperature and are completely miscible with water at room temperature, i.e. miscible with water at any proportion.

In a preferred embodiment the water-miscible organic solvents to be removed by the process of the present invention are selected from the group consisting of acetic acid, acetone, acetonitrile, methanol, ethanol, 1-propanol, 2-propanol, 1,4-dioxane, N,N-dimethylformamide, dimethylsulfoxide and tetrahydrofuran.

In a preferred embodiment of the present invention, the water-miscible organic solvents to be removed are selected from the group consisting of acetone, methanol, ethanol, 1-propanol, 2-propanol and mixtures thereof.

In a preferred embodiment of the present invention, the water-miscible organic solvents to be removed are selected from the group consisting of acetone, ethanol and 2-propanol and mixtures thereof, more preferably ethanol.

In a preferred embodiment, when Sugammadex or its salts, preferably Sugammadex sodium, comprises more than one water-miscible organic solvent selected from the group consisting of acetic acid, acetone, acetonitrile, methanol, ethanol, 1-propanol, 2-propanol, 1,4-dioxane, N,N-dimethylformamide, dimethylsulfoxide and tetrahydrofuran, the process of the present invention provides Sugammadex or its salts, preferably Sugammadex sodium, with a desired content of each one of said water-miscible organic solvents according to ICH guideline Q3C (R6).

In one particular embodiment of the present invention Sugammadex or its salts, preferably Sugammadex sodium, is stirred during the process, in order to favour the contact of the solid material with the gaseous medium at high relative humidity, so that the process is accelerated, and a more homogenous product is achieved.

In this particular embodiment, the process of the present invention can be performed in a paddle dryer, a rotary cone dryer, a tumble dryer, a rotary drum dryer, a tube bundle dryer, a stir tank reactor, a Nutsche filter, a centrifuge, a fluidized bed, a high shear mixer or a plate dryer.

In a preferred embodiment of the present invention Sugammdex or its salts, preferably Sugammadex sodium, is milled or micronized before the process of the invention is applied in order to favour the contact of the solid material with the gaseous medium at high relative humidity, so that the process is accelerated, and a more homogenous product is achieved.

In a preferred embodiment of the present invention Sugammadex or its salts, preferably Sugammadex sodium, is exposed to a relative humidity of 70% or higher, preferably to a relative humidity of 80% or higher, preferably to a relative humidity of 90% or higher, preferably to a relative humidity of from 95% to 100%, until Sugammadex or its salts, preferably Sugammadex sodium, has a water content of not less than 7% w/w, preferably of not less than 10% w/w, more preferably of not less than 15% w/w.

Certain relative humidities can be provided by any of the processes known in the art.

For example, at a laboratory scale, a certain relative humidity can be provided by using saturated solutions of different salts which are known to maintain particular values of relative humidities inside sealed containers. A relative humidity of about 100% may be provided by using an open recipient containing deionized water.

At a larger scale, e.g. industrial scale, a certain relative humidity can be provided by using a gas, for example air or nitrogen, having a controlled temperature and humidity in a container such as a dryer. Thus, the gas, for example air or nitrogen, can be bubbled into water, so that it takes certain humidity. This gas, for example air or nitrogen, is then preferably filtered through a cartridge filter before being entered into the dryer where Sugammadex or its salts, preferably Sugammadex sodium, has been previously charged. Alternatively, the humidity can be, for example, increased by producing some water vapor by heating liquid water with a resistence. Together with any means of control which activate cooling or heating liquid water at small intervals, the desired humidity can be achieved. In a particular embodiment, the process of the present invention further comprises at least one additional step comprising a vacuum drying step or a step of exposing Sugammadex or its salts, preferably Sugammadex sodium, to a gaseous medium, e.g. nitrogen, having low relative humidity, e.g. a relative humidity of 20% or lower.

In a particular embodiment, the process of the present invention comprises combining one or more steps of exposing Sugammadex or its salts, preferably Sugammadex sodium, to a relative humidity of 60% or higher, preferably to a relative humidity of 70% or higher, preferably to a relative humidity of 80% or higher, preferably to a relative humidity of 90% or higher, preferably to a relative humidity of from 95% to 100%, with one or more steps of vacuum drying of Sugammadex or its salts, preferably Sugammadex sodium.

In a particular embodiment, the step or steps of vacuum drying of the process of the present invention are performed at a temperature from 20° C. to 100° C., preferably from 40° C. to 80° C., more preferably about 70° C.

The term vacuum drying as used herein means drying under reduced pressure, i.e. a pressure lower than 760 mmHg.

In a preferred embodiment of the present invention the step or steps of vacuum drying is performed until Sugammadex or its salts, preferably Sugammadex sodium, has a water content of not more than 5% w/w, preferably of not more than 3% w/w.

The water content in % w/w of Sugammadex or its salts, preferably Sugammadex sodium, is preferably measured by Karl Fisher titration.

Sugammadex or its salts, preferably Sugammadex sodium, obtained according to the process of the invention has an ethanol content of not more than 5000 ppm. In another embodiment Sugammadex or its salts, preferably Sugammadex sodium, obtained according to the process of the invention contains only ethanol as organic solvent in an amount of not more than 5000 ppm.

Sugammadex or its salts, preferably Sugammadex sodium, obtained according to the process of the invention has, preferably, a 2-propanol content of not more than 5000 ppm. In another embodiment Sugammadex or its salts, preferably Sugammadex sodium, obtained according to the process of the invention contains only 2-propanol as organic solvent in an amount of not more than 5000 ppm.

Sugammadex or its salts, preferably Sugammadex sodium, obtained according to the process of the invention has, preferably, an acetone content of not more than 5000 ppm. In another embodiment Sugammadex or its salts, preferably Sugammadex sodium, obtained according to the process of the invention contains only acetone as organic solvent in an amount of not more than 5000 ppm.

In the embodiment of the present invention wherein Sugammadex or its salts, preferably Sugammadex sodium, obtained according to the process of the present invention, contains more than one water-miscible organic solvent selected from the group consisting of acetone, methanol, ethanol, 1-propanol or 2-propanol, Sugammadex or its salts, preferably Sugammadex sodium, obtained according to the process of the present invention has a residual content of each one of acetone, ethanol, 1-propanol and 2-propanol of not more than 5000 ppm and a content of methanol of not more than 3000 ppm.

In the embodiment of the present invention wherein Sugammadex or its salts, preferably Sugammadex sodium, obtained according to the process of the present invention, contains more than one water-miscible organic solvent selected from the group consisting of acetone, ethanol or 2-propanol, Sugammadex or its salts, preferably Sugammadex sodium, obtained according to the process of the present invention has a residual content of each one of acetone, ethanol and 2-propanol, preferably, of not more than 5000 ppm.

Sugammadex or its salts, preferably Sugammadex sodium, obtained according to the process of the present invention are used for the preparation of a medicament for the reversal of drug-induced neuromuscular block.

Sugammadex or its salts, preferably Sugammadex sodium, obtained according to the process of the present invention are preferably administered parenterally. The injection route can be intravenous, subcutaneous, intradermal, intramuscular, or intra-arterial. The intravenous route is the preferred one. The exact dose to be used will necessarily be dependent upon the needs of the individual subject to whom the medicament is being administered, the degree of muscular activity to be restored and the judgement of the anaesthetist/critical- care specialist.

Another aspect of the present invention involves pharmaceutical compositions comprising the Sugammadex or its salts, preferably Sugammadex sodium, obtained according to the process of the present invention. Preferably the pharmaceutical composition according to the present invention can be applied in the form of a solution, e.g. for use as an injection preparation.

Preferably the pharmaceutical compositions according to the present invention, preferably pharmaceutical compositions for use as an injection preparation, are prepared by mixing Sugammadex or its salts, preferably Sugammadex sodium, with water for injection. Preferably, the water for injection contains less than 100 ppm of oxygen, preferably less than 10 ppm of oxygen, more preferably less than 1 ppm of oxygen. The water for injection which contains less than 100 ppm of oxygen, preferably less than 10 ppm of oxygen, more preferably less than 1 ppm of oxygen, can be prepared by bubbling the water with an inert gas. The inert gas can be nitrogen or argon, preferably nitrogen.

The solution formed during the process of mixing Sugammadex or its salts, preferably Sugammadex sodium, with the water for injection having less than 100 ppm of oxygen, preferably less than 10 ppm of oxygen, more preferably less than 1 ppm of oxygen, is preferably bubbled with an inert gas, preferably nitrogen. The obtained solution is then preferably filtered and filled into vials. Finally, the vials can be sterilized by steam sterilisation upon heating in an autoclave, preferably at a temperature of about 121° C. for 15 minutes, although other temperature and time conditions, may also be used.

The pharmaceutical compositions according to the present invention are prepared by mixing Sugammadex or its salts, preferably Sugammadex sodium, obtained according to the process of the present invention with a pharmaceutically suitable liquid and optionally also with pharmaceutical suitable auxiliaries. e.g. as described in the standard reference, Gennaro et al., Remington’s Pharmaceutical Sciences, (18th ed., Mack Publishing Company, 1990, Part 8: Pharmaceutical Preparations and Their Manufacture; see especially Chapter 84 on “Parenteral preparations”, pp. 1545-1569; and Chapter 85 on “Intravenous admixtures”, pp. 1570-1580). Preferably the pharmaceutical compositions according to the present invention are prepared by mixing Sugammadex or its salts, preferably Sugammadex sodium, obtained according the process of the present invention with water for injection.

Alternatively, the pharmaceutical compositions of the present invention may be presented in unit-dose or multi-dose containers, for example sealed vials and ampoules, and may be stored in a freeze dried (lyophilised) condition requiring only the addition of the sterile liquid carrier, for example, water for injection prior to use.

In a further aspect the invention relates to a kit for providing neuromuscular block and its reversal comprising (a) a neuromuscular blocking agent, and (b) Sugammadex or its salts, preferably Sugammadex sodium, prepared according to the process of the present invention.

A preferred kit, according to the invention, contains a Sugammadex or its salts, preferably Sugammadex sodium, prepared according to the process of the present invention and a neuromuscular blocking agent which is selected from the group consisting of rocuronium, vecuronium, pancuronium, rapacuronium, mivacurium, atracurium, (cis) atracurium, tubocurarine and suxamethonium.

The term “about” when used in the present invention preceding a number and referring to it, is meant to designate any value which lies within the range defined by the number ±10% of its value, preferably a range defined by the number ±5%, more preferably range defined by the number ±2%, still more preferably a range defined by the number ±1%. For example “about 10” should be construed as meaning within the range of 9 to 11, preferably within the range of 9.5 to 10.5, more preferably within the range of 9.8 to 10.2, and still more preferably within the range of 9.9 to 10.1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples further illustrate the invention but, of course, should not be construed as in any way limiting its scope.

### EXAMPLES

General experimental conditions:

### GC Method Used for Determining Ethanol Content

Equipment: A gas chromatograph equipped with a DB-624 capillary column (Agilent, 75 m. x 0.53 mm. i.d., 3 µm film thickness) or equivalent was used. The chromatograph was equipped with an FID detector and a Head Space injection device. An agilent 7890A chromatograph with a Head Space Agilent G1888 was used.

Chromatographic conditions: The oven temperature was set at 40° C. for about 10 minutes, then raised to 75° C. with a ramp of 2° C. per minute and maintained at 75° C. for 10 minutes, then raised again to 240° C. with a ramp of 30° C. per minute and maintained at 240° C. for 5 minutes. The injector temperature was set at 220° C. and the detector temperature was set at 250° C. Helium was used as carrier gas at a pressure of 8 psi and with a split ratio of 2:1.

Headspace conditions: The temperatures of loop and transfer line were set at 100° C. Each sample was heated for 30 minutes at 85° C. After heating, vials were pressurized with helium at 18 psi for 0.3 minutes. The sample loop was filled for 0.15 min (loop volume = 1 mL), equilibrated for 0.05 min and then injected for 0.5 minutes.

### Preparation of Solutions

Stock solution of ethanol: A solution containing 1003.30 pg/mL of ethanol in water was prepared by dissolving quantitatively 127.0 µL of ethanol in a 100.0 mL volumetric flask and diluting with water to volume.

Standard solution of ethanol: 25.0 mL of Stock solution of ethanol were diluted quantitatively to 50.0 mL of water. This solution contained 501.65 µg/mL of ethanol corresponding to 25083 ppm of ethanol in Test specimen.

Test solution: A solution of about 100 mg of Sugammadex sodium in 5.0 mL of water was prepared by triplicate.

Procedure: Vials of 20 mL capacity, suitable for head space injection, were prepared. 5.0 mL of water were introduced to each one of three vials, 5.0 mL of Standard solution of ethanol were introduced to each one of six vials and 5.0 mL of Test solution were introduced to each one of three vials.

The vials were sealed with suitable crimp caps and analyzed by headspace using the described conditions.

In the above conditions the retention time of ethanol was about 8.7 minutes.

### System Suitability

The following requirements were to be fulfilled:
Maximal permitted relative standard deviation for 6 replicate injections of Standard solution of ethanol was not more than 15.0 %.
The symmetry factor (or Tailing factor) of ethanol peak in the Standard solution was between 0.8 and 2.5.

### Calculation

The amount of ethanol (ppm) in Test solution was calculated by using the following formula:

$$\text{Content of ethanol} \left(\text{ppm}\right) = \frac{{A}_{T}}{{A}_{S}} \times \frac{5 \times {C}_{s}}{w}$$

Where:
A_{T}: Area response of ethanol peak in Test solution.
Aₛ: Area response of ethanol peak in the suitable Standard solution of ethanol.
Cₛ: Concentration in pg/mL of ethanol in the suitable Standard solution of ethanol.
W: Weight (g) of Sugammadex sodium used to prepare Test solution.
5: Volume (mL) used to dissolve Test solution.

The final value of the content of ethanol (ppm) is calculated as the average of the three results obtained for each one of the triplicates.

### GC Method Used for Determining 2-Propanol Content

Equipment: A gas chromatograph equipped with a DB-624 capillary column (Agilent, 75 m. x 0.53 mm. i.d., 3 µm film thickness) or equivalent was used. The chromatograph was equipped with a FID detector and a Head Space injection device. An agilent 7890A chromatograph with a Head Space Agilent 7697A was used.

Chromatographic conditions: The oven temperature was set at 40° C. for about 10 minutes, then raised to 75° C. with a ramp of 2° C. per minute and maintained at 75° C. for 10 minutes, then raised again to 240° C. with a ramp of 30° C. per minute and maintained at 240° C. for 5 minutes. The injector temperature was set at 220° C. and the detector temperature was set at 250° C. Helium was used as carrier gas at a pressure of 8 psi and with a split ratio of 2:1.

Headspace conditions: The temperatures of loop and transfer line were set at 100° C. Each sample was heated for 30 minutes at 85° C. After heating, vials were pressurized with helium at 18 psi for 0.3 minutes. The sample loop was filled for 0.15 min (loop volume = 1 mL), equilibrated for 0.05 min and then injected for 0.5 minutes.

### Preparation of Solutions

Stock solution of 2-propanol: A solution containing 2009.60 µg/mL of 2-propanol in water was prepared by dissolving quantitatively 64.0 µL of 2-propanol in a 25.0 mL volumetric flask and diluting with water to volume.

Intermediate standard solution of 2-propanol: 5.0 mL of Stock solution of 2-propanol were diluted quantitatively to 50.0 mL of water. This solution contained 200.96 µg/mL of 2-propanol corresponding to 10048 ppm of 2-propanol in Test specimen.

Standard solution of 2-propanol: 2.0 mL of Intermediate solution of 2-propanol were diluted quantitatively to 200.0 mL of water. This solution contained 2.01 µg/mL of 2-propanol corresponding to 101 ppm of 2-propanol in Test specimen.

Test solution: A solution of about 100 mg of Sugammadex sodium in 5.0 mL of water was prepared by triplicate.

Procedure: Vials of 20 mL capacity, suitable for head space injection, were prepared. 5.0 mL of water were introduced to each one of three vials, 5.0 mL of Standard solution of 2-propanol were introduced to each one of six vials and 5.0 mL of Test solution were introduced to each one of three vials.

The vials were sealed with suitable crimp caps and analyzed by headspace using the described conditions.

In the above conditions the retention time of 2-propanol was about 10.4 minutes.

### System Suitability

The following requirements were to be fulfilled:
Maximal permitted relative standard deviation for 6 replicate injections of Standard solution of 2-propanol was not more than 15.0 %.
The symmetry factor (or Tailing factor) of 2-propanol peak in the Standard solution was between 0.8 and 2.5.

### Calculation

The amount of 2-propanol (ppm) in Test solution was calculated by using the following formula:

$$\text{Content of 2-propanol} \left(\text{ppm}\right) = \frac{{A}_{T}}{{A}_{S}} \times \frac{5 \times {C}_{S}}{w}$$

Where:
A_{T}: Area response of 2-propanol peak in Test solution.
Aₛ: Area response of 2-propanol peak in the Standard solution of 2-propanol
Cₛ: Concentration in pg/mL of 2-propanol in the Standard solution of 2-propanol.
W: Weight (g) of Sugammadex sodium used to prepare Test solution.
5: Volume (mL) used to dissolve Test solution.

The final value of the content of 2-propanol (ppm) is calculated as the average of the three results obtained for each one of the triplicates.

### GC Method Used for Determining Acetone Content

Equipment: A gas chromatograph equipped with a DB-624 capillary column (Agilent, 75 m. x 0.53 mm. i.d., 3 µm film thickness) or equivalent was used. The chromatograph was equipped with a FID detector and a Head Space injection device. An Agilent 7890A chromatograph with a Head Space Agilent G1888 was used.

Chromatographic conditions: The oven temperature was set at 40° C. for about 10 minutes, then raised to 75° C. with a ramp of 2° C. per minute and maintained at 75° C. for 10 minutes, then raised again to 240° C. with a ramp of 30° C. per minute and maintained at 240° C. for 5 minutes. The injector temperature was set at 220° C. and the detector temperature was set at 250° C. Helium was used as carrier gas at a pressure of 8 psi and with a split ratio of 2:1.

Headspace conditions: The temperatures of loop and transfer line were set at 100° C. Each sample was heated for 30 minutes at 85° C. After heating, vials were pressurized with helium at 18 psi for 0.3 minutes. The sample loop was filled for 0.15 min (loop volume = 1 mL), equilibrated for 0.05 min and then injected for 0.5 minutes.

### Preparation of Solutions

Stock solution of acetone: A solution containing 102.57 pg/mL of acetone in water was prepared by dissolving quantitatively 13 µL of acetone in a 100.0 mL volumetric flask and diluting with water to volume.

Intermediate standard solution of acetone: 10.0 mL of Stock solution of acetone were diluted quantitatively to 50.0 mL of water. This solution contained 20.51 pg/mL of acetone corresponding to 1026 ppm of acetone in Test specimen.

Standard solution of acetone: 10.0 mL of Intermediate standar solution of acetone were diluted quantitatively to 100.0 mL of water. This solution contained 2.05 pg/mL of acetone corresponding to 103 ppm of acetone in Test specimen.

Test solution: A solution of about 100 mg of Sugammadex sodium in 5.0 mL of water was prepared by triplicate.

Procedure: Vials of 20 mL capacity, suitable for head space injection, were prepared. 5.0 mL of water were introduced to each one of three vials, 5.0 mL of Standard solution of acetone were introduced to each one of six vials and 5.0 mL of Test solution were introduced to each one of three vials.

The vials were sealed with suitable crimp caps and analyzed by headspace using the described conditions.

In the above conditions the retention time of acetone was about 10.1 minutes.

### System Suitability

The following requirements were to be fulfilled:
Maximal permitted relative standard deviation for 6 replicate injections of Standard solution of acetone was not more than 15.0 %.
The symmetry factor (or Tailing factor) of acetone peak in the Standard solution was between 0.8 and 2.5.

### Calculation

The amount of acetone (ppm) in Test solution was calculated by using the following formula:

$$\text{Content of acetone} \left(\text{ppm}\right) = \frac{{A}_{T}}{{A}_{S}} \times \frac{5 \times {C}_{s}}{w}$$

Where:
A_{T}: Area response of acetone peak in Test solution.
Aₛ: Area response of acetone peak in the Standard solution of acetone
Cₛ: Concentration in pg/mL of acetone in the Standard solution of acetone
W: Weight (g) of Sugammadex sodium used to prepare Test solution.
5: Volume (mL) used to dissolve Test solution.

The final value of the content of acetone (ppm) is calculated as the average of the three results obtained for each one of the triplicates.

### Reference Example 1

About 6 g of Sugammadex sodium having a residual content of 16844 ppm of ethanol were placed in a laboratory dish and were introduced into a closed laboratory plate dryer under vacuum. The sample was exposed to five successive cycles where the temperature was set to 70° C. for 8 hours and to 25° C. for 16 hours.

The sample was homogenized daily before starting the step at 25° C. and aliquots were taken for analysis of residual ethanol content. The results are depicted in table 1 below:

TABLE 1

| | EtOH, ppm |
|---|---|
| Initial sample | 16844 |
| After 1 day | 16997 |
| After 2 days | 16093 |
| After 3 days | 16369 |
| After 4 days | 16255 |
| After 5 days | 16209 |

As it is shown in the table 1 above, after five days of keeping Sugammadex sodium under vacuum alterning temperatures of 70° C. during 8 hours and of 25° C. during 16 hours, the amount of ethanol is not substantially decreased. The results indicate that the content of ethanol decreases very little (approximately around 600 ppm).

### Reference Example 2

10 g of Sugammadex sodium were dissolved in 61.5 mL of water at 25-30° C. pH was adjusted in the range of 9.0-9.95 with aqueous NaOH 1.5 M, and 12.3 mL of 2-propanol were added. The resulting solution was then added over 61.26 mL of 2-propanol at 20-25° C. Additional 122.5 mL 2-propanol were added. The resulting suspension was stirred for 1h and the resulting solid collected by filtration, washed with 24.5 mL of 2-propanol, and dried at 70-75° C. in a vacuum oven for 15 hours. After drying, the amount of residual 2-propanol was of 26815 ppm.

Therefore, after drying Sugammadex sodium obtained from 2-propanol and water during 15 hours at 70-75° C. under vacuum, the amount of residual 2-propanol is still of 26815 ppm, showing that standard drying conditions do not allow by far to reduce the residual 2-propanol content to values according to ICH guideline Q3C (R6).

### Reference Example 3

10 g of Sugammadex sodium were dissolved in 61.5 mL of water at 25-30° C. pH was adjusted in the range of 9.0-9.95 with aqueous NaOH 1.5 M, and 12.3 mL of acetone were added. The resulting solution was then added over 61.26 mL of acetone at 20-25° C. Additional 122.5 mL acetone were added. The resulting suspension was stirred for 1h and the resulting solid collected by filtration, washed with 24.5 mL of acetone, and dried at 70-75° C. in a vacuum oven for 15 hours. After drying, the amount of residual acetone was of 21107 ppm.

Therefore, after drying Sugammadex sodium obtained from acetone and water during 15 hours at 70-75° C. under vacuum, the amount of residual acetone is still of 21107 ppm, showing that standard drying conditions do not allow by far to reduce the residual acetone content to values according to ICH guideline Q3C (R6).

Example 1: drying of Sugammadex sodium wetted with ethanol by exposing Sugammadex sodium to a relative humidity of 84%.

About 6 g of sugammadex sodium having a residual content of 19249 ppm of ethanol were placed into a closed laboratory plate dryer together with an open recipient containing a saturared aqueous solution of potassium chloride, at 25° C. and under atmospheric pressure, so that the relative humidity was maintained constant at a value of 84%.

The sample was homogenized and a small aliquot taken daily during 4 days. These aliquots were analyzed for residual ethanol content. The results are depicted in table 2 below:

TABLE 2

| | EtOH, ppm |
|---|---|
| Initial sample | 19249 |
| After 1 Day | 17478 |
| After 2 Days | 13379 |
| After 3 Days | 9899 |
| After 4 Days | 6915 |

As shown in the table 2 above the amount of ethanol is drastically reduced, after exposing sugammadex sodium at 25° C. and 84% of relative humidity for four days.

Example 2: drying of Sugammadex sodium wetted with ethanol combining vacuum drying steps and steps of exposing Sugammadex sodium to a relative humidity of 100%.

A sample of Sugammadex sodium having a residual content of 14149 ppm of ethanol was introduced into a closed laboratory plate dryer together with an open recipient containing deionized water, at 25° C. and under atmospheric pressure, so that the relative humidity was maintained constant at a value of 100% for the subsequent 16 hours. After this time, the recipient containing deionized water was removed, vacuum was applied, and the temperature was set to 70° C. for the next 8 hours. Then, the temperature was cooled to 25° C., the sample was homogenized, and an aliquot was taken for analysis of residual ethanol content.

The resulting sample was subjected to the same cycle described above of exposing at 25° C. under atmospheric pressure to a relative humidity of 100% for 16 hours, followed by heating at 70° C. under vacuum for 8 hours, for a total of 3 days. Aliquots were taken daily for analysis of residual ethanol content. The results are depicted in table 3 below:

TABLE 3

| | EtOH, ppm |
|---|---|
| Initial sample | 14149 |
| After 1 Day | 13062 |
| After 2 Days | 7802 |
| After 3 Days | 2963 |

As it is shown in the table 3 above the amount of ethanol is drastically reduced, after performing cycles of exposure to a relative humidity of 100% at 25° C. and vacuum drying at 70° C. for three days.

Example 3: drying of Sugammadex sodium wetted with ethanol combining vacuum drying steps and steps of exposing Sugammadex sodium to a relative humidity of 94%.

A sample of Sugammadex sodium having a residual content of 20483 ppm of ethanol was introduced into a closed laboratory plate dryer together with an open recipient containing a saturated aqueous solution of potassium nitrate, at 25° C. and under atmospheric pressure, so that the relative humidity was maintained constant at a value of 94% for the subsequent 16 hours. After this time, the recipient containing a saturated aqueous solution of potassium nitrate was removed, vacuum was applied, and the temperature was set to 70° C. for the next 8 hours. Then, the temperature was cooled to 25° C., the sample was homogenized, and an aliquot was taken for analysis of residual ethanol content.

The resulting sample was subjected to the same cycle described above of exposing at 25° C. under atmospheric pressure to a relative humidity of 94% for 16 hours, followed by heating at 70° C. under vacuum for 8 hours, for a total of 4 days. Aliquots were taken daily for analysis of residual ethanol content. The results are depicted in table 4 below:

TABLE 4

| | EtOH, ppm |
|---|---|
| Initial sample | 20483 |
| After 1 Day | 16905 |
| After 2 Days | 10752 |
| After 3 Days | 5313 |
| After 4 Days | 1259 |

As it is shown in the table 4 above the amount of ethanol is drastically reduced, after performing cycles of exposure to a relative humidity of 94% at 25° C. and vacuum drying at 70° C. for four days.

Example 4: drying of Sugammadex sodium wetted with ethanol combining vacuum drying steps and steps of exposing Sugammadex sodium to a relative humidity of 75%.

A sample of Sugammadex sodium having a residual content of 20647 ppm of ethanol was introduced into a closed laboratory plate dryer together with an open recipient containing a saturated aqueous solution of sodium chloride, at 25° C. and under atmospheric pressure, so that the relative humidity was maintained constant at a value of 75% for the subsequent 16 hours. After this time, the recipient containing a saturated aqueous solution of sodium chloride was removed, vacuum was applied, and the temperature was set to 70° C. for the next 8 hours. Then, the temperature was cooled to 25° C., the sample was homogenized, and an aliquot was taken for analysis of residual ethanol content.

The resulting sample was subjected to the same cycle described above of exposing at 25° C. under atmospheric pressure to a relative humidity of 75% for 16 hours, followed by heating at 70° C. under vacuum for 8 hours, for a total of 4 days. Aliquots were taken daily for analysis of residual ethanol content. The results are depicted in table 5 below:

TABLE 5

| | EtOH, ppm |
|---|---|
| Initial sample | 20647 |
| After 1 Day | 18911 |
| After 2 Days | 16174 |
| After 3 Days | 13074 |
| After 4 Days | 9507 |

As it is shown in the table 5 above the amount of ethanol is reduced, after performing cycles of exposure to a relative humidity of 75% at 25° C. and vacuum drying at 70° C. for four days.

Example 5: drying of Sugammadex sodium wetted with ethanol combining a vacuum drying step and a step of exposing Sugammadex sodium to a relative humidity of 100% under agitation.

A sample of Sugammadex sodium having a residual content of 19414 ppm of ethanol was introduced into a closed laboratory plate dryer together together with an open recipient containing deionized water, at 25° C. and under atmospheric pressure, so that the relative humidity was maintained constant at a value of 100% for 22 hours. The sample was periodically, i.e. every hour during the first eight hours, agitated and homogenized.

After these 22 hours, a sample was taken for KF analysis showing that the water content was of 15.41 % and the rest was dried at 75° C. under vacuum for 8h, yielding Sugammadex sodium with a content in ethanol of 4146 ppm and a water content of 2.15%.

Example 6: drying of Sugammadex sodium wetted with 2-propanol combining vacuum drying steps and steps of exposing Sugammadex sodium to a relative humidity of 94%.

A sample of Sugammadex sodium having a residual content of 35007 ppm of 2-propanol was introduced into a closed laboratory plate dryer together with an open recipient containing a saturared aqueous solution of potassium nitrate, at 25° C. and under atmospheric pressure, so that the relative humidity was maintained constant at a value of 94% for the subsequent 16 hours. After this time, the recipient containing a saturared aqueous solution of potassium nitrate was removed, vacuum was applied, and the temperature was set to 70° C. for the next 8 hours. Then, the temperature was cooled to 25° C., the sample was homogenized, and an aliquot was taken for analysis of residual 2-propanol content.

The resulting sample was subjected to the same cycle described above of exposing at 25° C. under atmospheric pressure to a relative humidity of 94% for 16 hours, followed by heating at 70° C. under vacuum for 8 hours, for a total of 4 days. Aliquots were taken daily for analysis of residual 2-propanol content. The results are depicted in table 6 below:

TABLE 6

| | 2-propanol, ppm |
|---|---|
| Initial sample | 35007 |
| After 1 Day | 29416 |
| After 2 Days | 23127 |
| After 3 Days | 18960 |
| After 4 Days | 11559 |

As it is shown in the table 6 above the amount of 2-propanol is reduced, after performing cycles of exposure to a relative humidity of 94% at 25° C. and vacuum drying at 70° C. for four days.

Example 7: drying of Sugammadex sodium wetted with acetone combining vacuum drying steps and steps of exposing Sugammadex sodium to a relative humidity of 94%.

A sample of Sugammadex sodium having a residual content of 27070 ppm of acetone was introduced into a closed laboratory plate dryer together with an open recipient containing a saturared aqueous solution of potassium nitrate, at 25° C. and under atmospheric pressure, so that the relative humidity was maintained constant at a value of 94% for the subsequent 16 hours. After this time, the recipient containing a saturared aqueous solution of potassium nitrate was removed, vacuum was applied, and the temperature was set to 70° C. for the next 8 hours. Then, the temperature was cooled to 25° C., the sample was homogenized, and an aliquot was taken for analysis of residual acetone content.

The resulting sample was subjected to the same cycle described above of exposing at 25° C. under atmospheric pressure to a relative humidity of 94% for 16 hours, followed by heating at 70° C. under vacuum for 8 hours, for a total of 4 days. Aliquots were taken daily for analysis of residual acetone content. The results are depicted in table 7 below:

TABLE 7

| | Acetone, ppm |
|---|---|
| Initial sample | 27070 |
| After 1 Day | 19242 |
| After 2 Days | 17207 |
| After 3 Days | 11061 |
| After 4 Days | 5723 |

As it is shown in the table 7 above the amount of acetone is drastically reduced, after performing cycles of exposure to a relative humidity of 94% at 25° C. and vacuum drying at 70° C. for four days.

1. A process for removing water-miscible organic solvents from Sugammadex or its salts, preferably from Sugammadex sodium, which comprises exposing Sugammadex or its salts to a relative humidity of 70% or higher, wherein at least one of the water-miscible organic solvents to be removed is selected from the group consisting of acetic acid, acetone, acetonitrile, ethanol, 1-propanol, 2-propanol, 1,4-dioxane, N,N-dimethylformamide, dimethylsulfoxide and tetrahydrofuran.

2. The process according to claim 1, wherein the relative humidity is of 80% or higher.

3. The process according to claim 2, wherein the relative humidity is of 90% or higher.

4. The process according to claim 3, wherein the relative humidity is of from 95% to 100%.

5. The process according to claim 1, wherein the process is performed at a temperature from 0° C. to 100° C., preferably from 20° C. to 60° C., more preferably from 20° C. to 30° C., even more preferably about 25° C.

6. (canceled)

7. The process according to claim 1, wherein the at least one water-miscible organic solvent to be removed is selected from the group consisting of acetone, ethanol and 2-propanol, preferably ethanol.

8. The process according to claim 1, wherein Sugammadex or its salts, preferably Sugammadex sodium, is stirred during the process.

9. The process according to claim 1, wherein Sugammadex or its salts, is exposed to a relative humidity of 70% or higher, until Sugammadex or its salts has a water content of not less than 7% w/w.

10. The process according to claim 1, wherein the process comprises combining one or more steps of exposing Sugammadex or its saltsto a relative humidity of 70% or higher with one or more steps of vacuum drying of Sugammadex or its salts.

11. The process according to claim 10, wherein the vacuum drying step is performed at a temperature from 20° C. to 100° C.

12. The process according to claim 10, wherein the vacuum drying step is performed until Sugammadex or its salts, has a water content of not more than 5% w/w.

13. The process according to claim 1, wherein the obtained Sugammadex or its salts has an ethanol content, an acetone content and/or a 2-propanol content of not more than 5000 ppm.

14. A pharmaceutical composition comprising Sugammadex or its salts obtained according to the process as defined in claim 1.

15. A method for reversing a neuromuscular blocking drug in a subject comprising administering to the subject the pharmaceutical composition of claim 14.

16. The process according to claim 9, wherein Sugammadex or its salts is exposed to a relative humidity of 90% or higher until Sugammadex or its salts has a water content of not less than 15% w/w.

17. The process according to claim 10, wherein the process comprises combining one or more steps of exposing Sugammadex or its salts to a relative humidity of 90% or higher with one or more steps of vacuum drying of Sugammadex or its salts.

18. The process according to claim 17, wherein the vacuum drying step is performed at a temperature of about 70%.

19. The process according to claim 17, wherein the vacuum drying step is performed until Sugammadex or its salts has a water content of not more than 3% w/w.
